(19) United States
(12) Patent Application Publication
(10) Pub. No.: US 2012/0094919 A1
(43) Pub. Date: Apr. 19, 2012

(54) USE OF TRIPEPTIDES

(76) Inventors: Remo Gräub, Bern (CH); Marc Heidl, Lorrach (DE); Dominik Imfeld, Munchenstein (CH); Eike Müller, Kaiseraugst (CH); Peter Wikstroem, Gipf-Oberfrick (CH); Hugo Ziegler, Witterswil (CH)

(21) Appl. No.: 13/254,564

(22) PCT Filed: Mar. 16, 2010
(86) PCT No.: PCT/EP2010/053336

§ 371 Date: Dec. 23, 2011

(30) Foreign Application Priority Data
Mar. 16, 2009 (EP) 09155204.2
Jul. 23, 2009 (EP) 09166218.9

(51) Int. Cl. A61K 8/64; A61Q 19/00

(52) U.S. Cl. 514/18.8

(57) ABSTRACT

The present invention relates to the use of tripeptide derivatives for tightening, firming and/or moisturizing skin. Furthermore, the invention relates to a method for stimulating the synthesis of glycosaminoglycans containing a D-glucosamine and/or N-acetyl-D-glucosamine residue and/or proteoglycans by fibroblasts and/or keratinocytes such as in particular the synthesis of Hyaluronic acid and/or of the proteoglycans Decorin and/or Lumican.

The present invention relates to the use of certain specific tripeptide derivatives for tightening, firming and/or moisturizing skin. Furthermore, the invention relates to a method for stimulating the synthesis of glycosaminoglycans containing a D-glucosamine and/or N-acetyl-D-glucosamine residue and/or proteoglycans by fibroblasts and/or keratinocytes such as in particular the synthesis of Hyaluronan (Hyaluronic acid) and/or the proteoglycans Decorin and/or Lumican.

Skin consists of a set of cells grouped together in the form of supple, resistant tissue covering the whole of the body. The main role played by skin is as a protective barrier against external factors at the same time as allowing certain exchanges between the interior and exterior environment. It is the site of many metabolic processes that are regulated by the organism's physiological conditions and environmental conditions. Skin consists of two adjacent layers, the epidermis and the dermis, to which subcutaneous tissue is attached.

The epidermis, whose principal role is to protect the body, is the uppermost layer of the skin and gives the skin its impermeability and resistance. It is renewed approximately every four weeks. While different cell types co-exist in the epidermis, the keratinocytes constitute the main cell type (90%). Their characteristic activity is that of keratin synthesis which make up 95% of the epidermis' total proteins. The keratins, fibrous and water-insoluble proteins, are constituents of the corneal layer of the epidermis which protects the skin against harmful external factors (heat, cold, dehydration).

The epidermis is connected to the dermis through a zone called the dermo-epidermal junction or epidermal basal membrane. This structure provides adhesion of the dermis to the epidermis and has a mechanical support role which is partly responsible for skin tonicity. It is made up of both basal keratinocytes and dermal fibroblasts.

The dermis, the skin's inner layer, is a fibro-elastic conjunctive tissue comprised of cells (fibroblasts) dispersed in a complex medium called the extracellular matrix. This matrix consists of collagen, elastin fibers, glycoproteins and proteoglycans (PGs). Glycosaminoglycans (GAGs) in its free form (i.e. not attached to a protein) are also found in the extracellular matrix.

Glycosaminoglycans (GAG) are polymers formed of disaccharide units. Hyaluronic acid may be mentioned first of all among the GAG most frequently found in human skin, being present in the greatest abundance. Also present are chondroitin 4-sulfate and 6-sulfate, dermatan sulfate and, in small amounts, heparin and heparan sulfate. The disaccharide units of GAG are formed of a hexosamine (D-glucosamine or D-galactosamine), fairly often sulfated, alternating with an uronic acid (D-glucuronic or L-iduronic acid).

GAG are generally covalently bonded to proteins to form proteoglycans such as e.g. Lumican or Decorin which both belong to the small leucine-rich proteoglycan (SLRP) family. Among the known GAG, only Hyaluronic acid, mentioned above, is not synthesized bonded to a central protein.

Proteoglycans are complex macromolecules consisting of a branched central protein trunk, or protein network, to which numerous carbohydrate side chains known as glycosaminoglycans are attached. The structure of proteoglycans is e.g. described in ‘The Journal of investigative dermatology (1982), 79, Suppl. 1, 31s-37s’.

It is well known that through their property of associating strongly with water molecules and forming gels, GAGs as well as proteoglycans ensure the moisturization of the dermis and epidermis. A well-moisturized skin guarantees a good appearance and a satisfactory physiological and functional state, with good mechanical properties in particular.

Specifically, over the course of aging, the fibroblasts and keratinocytes produce less and less PGs and GAGs and their synthesis is imperfect. This results in a considerable disorganization: the deposition of GAGs on the protein skeleton forming the PG is abnormal, the consequence of this is a reduced avidity for water of these PGs and thus a reduction in the moisturization and tonicity of tissues which is inter alia reflected by a loss of suppleness of the skin.

Thus, there is an ongoing need for active agents whose effects are directed towards maintaining the level of PGs such as Lumican or Decorin and GAGs such as Hyaluronan in the skin and thus are suitable to be used for tightening, firming and/or moisturizing skin. As a result the skin appears lifted, the skin tonicity is improved, skin sagging is prevented or decreased and the suppleness and elasticity of the skin is maintained or restored (remodeling effect). Furthermore, cicatrization is facilitated, epidermal micro-traumas can be repaired and stretch marks are reduced.

Surprisingly, it has been found that certain specific tripeptide derivatives are capable of increasing the synthesis of Hyaluronan (Hyaluronic acid) and/or the proteoglycans Decorin and/or Lumican by human fibroblasts and are thus particularly useful in treating the skin conditions outlined above which, as discussed, are due to an insufficiency in the production of these GAGs and PGs. This has furthermore been illustrated in a vivo study showing an improvement of the skin tonicity and skin firmness as well a remodeling effect i.e. an effect on sagging and double chin after topical application of a cosmetic composition comprising such tripeptide derivatives.

Thus, the invention relates to the use of tripeptide derivatives of the general formula (I)

R1-A-B-C-R2 (I),

wherein
R1 means palmitoyl or tetradecylaminocarbonyl;
R2 means OH or NH-hexadecyl;
A means argininyl, lysyl, ornithyl or 2,4-diaminobutyroyl;
B means valyl, leucyl, isoleucyl or norvalyl and
C means argininyl, lysyl or 2,4-diaminobutyroyl
and the dermatologically tolerated salts thereof for tightening, firming and/or moisturizing the skin.

In particular, the tripeptide derivatives according to the invention are suitable for lifting the skin, preventing or decreasing skin sagging, maintaining or restoring the suppleness and elasticity of the skin (remodeling effect), facilitating cicatrization and repairing epidermal micro-traumas and/or reducing stretch marks.

In another embodiment, the invention relates to a method for stimulating the synthesis of glycosaminoglycans containing a D-glucosamine and/or N-acetyl-D-glucosamine residue and/or proteoglycans by fibroblasts and/or keratinocytes comprising administering to an individual in need of such treatment an effective amount of at least one tripeptide derivative corresponding to formula (I) as outlined above. In particular the invention relates to a method for stimulating the synthesis of Hyaluronan (Hyaluronic acid) and/or the proteoglycans Decorin and/or Lumican. Said method is in particular suitable for the treatment of subjects suffering from an insufficiency of glycosaminoglycan synthesis and/or proteoglycan synthesis, particularly of hyaluronic acid, Decorin and/or Lumican synthesis as discussed above, in order to correct the adverse effects of said insufficiency by improving the tightness and firmness of the skin and/or by improving the moisturization of the skin. Consequently, said method is in particular suitable for lifting the skin, for the treatment or co-treatment of skin sagging, for maintaining or restoring the suppleness and elasticity of the skin, for facilitating cicatrization, for repairing epidermal micro-traumas or for reducing stretch marks.

Furthermore, the invention relates to the use of at least one tripeptide derivative corresponding to formula (I) in which R1 is palmitoyl or tetradecylaminocarbonyl; R2 is OH or NH-hexadecyl; A is argininyl, lysyl, ornithyl or 2,4-diaminobutyroyl; B is valyl, leucyl, isoleucyl or norvalyl and C is argininyl, lysyl or 2,4-diaminobutyroyl or the dermatologically tolerated salts thereof as outlined above for the stimulation of the synthesis of Hyaluronan (Hyaluronic acid).

The invention further relates to any method of therapeutic treatment of the skin by which it is desired to stimulate glycosaminoglycan synthesis, particularly hyaluronic acid synthesis.

This invention also encompasses the optical isomers of the tripeptide derivatives corresponding to formula (I), and also to the physiologically acceptable salts of these derivatives.

The compounds of formula (I) together with acids can form mono- or polyvalent, homogeneous or mixed salts, e.g. with inorganic acids, such as hydrochloric acid, hydrobromic acid, sulfuric acid or phosphoric acid; or with appropriate carboxylic acids, e.g. aliphatic mono- or dicarboxylic acids, such as formic acid, acetic acid, trifluoroacetic acid, trichloroacetic acid, propionic acid, glycolic acid, succinic acid, fumaric acid, malonic acid, maleic acid, oxalic acid, phthalic acid, citric acid, lactic acid or tartaric acid; or with aromatic carboxylic acids, such as benzoic acid or salicylic acid; or with aromatic-aliphatic carboxylic acids, such as mandelic acid or cinnamic acid; or with heteroaromatic carboxylic acids, such as nicotinic acid; or with aliphatic or aromatic sulfonic acids, such as methanesulfonic acid or toluenesulfonic acid. Preferred are “dermatologically tolerated salts”.

According to the invention, the tripeptide derivatives corresponding to formula (I) may be used alone or as a mixture in any proportion.

According to one preferred embodiment of the invention, the tripeptide derivatives corresponding to formula (I) that are used are those for which A and C, which may be identical or different, have the definitions of lysyl or 2,4-diaminobutyroyl.

According to another preferred embodiment of the invention, the tripeptide derivatives corresponding to formula (I) that are used are those for which R2 represents OH.

According to another preferred embodiment of the invention, the tripeptide derivatives corresponding to formula (I) are chloride, acetate or trifluoroacetate salts, in particular trifluoroacetate salts.

Among the tripeptide derivatives of formula (I) used according to the invention, the ones that are preferred are:

### Palm-Lys-Val-Lys-OH

### Palm-Orn-Val-Lys-OH

### Palm-Lys-Leu-Lys-OH

### Palm-Lys-Ile-Dab-OH

### Palm-Lys-Nva-Dab-OH

### Palm-Lys-Leu-Dab-OH

### Palm-Lys-Val-Dab-NH-Hexadecyl

### Tetradecyl-NH—C(O)-Dab-Val-Dab-OH

### Tetradecyl-NH—C(O)-Lys-Ile-Dab-OH

### Tetradecyl-NH—C(O)-Arg-Val-Arg-OH,

wherein Palm means palmitoyl and Dab means 2,4-diaminobutyroyl.

Among the tripeptide derivatives of formula (I) used according to the invention, the most preferred one is: Tetradecyl-NH—C(O)-Dab-Val-Dab-OH, in particular in the form of the trifluoroacetate salt.

Preferably, all amino acids in the tripeptide derivatives according to the invention are L-configurated.

The tripeptide derivatives corresponding to formula (I) and the composition thereof are known in the patent literature and described in WO 2004/099237.

The tripeptide derivatives according to the invention may be used in any desired application form suitable for tightening, firming and/or moisturizing skin such as e.g. in topical compositions. However, the tripeptide derivatives according to the invention are also suitable to be encapsulated in nanoparticles such as liposomes, nanosomes, cyclodextrins, which subsequently may be incorporated into the desired application form.

Preferably, an effective amount of at least one tripeptide derivative with the definitions and preferences as given above is incorporated into a topical composition further comprising a cosmetically acceptable carrier.

Such topical compositions are in particular suitable for tightening, firming and/or moisturizing skin such as particularly for lifting the skin, preventing or decreasing skin sagging, maintaining or restoring the suppleness and elasticity of the skin (remodeling effect), facilitating cicatrization and repairing epidermal micro-traumas and/or reducing stretch marks.

The term “effective amount” means generally at least 0.00001% by weight of the topical composition. Preferably, the compositions contain the tripeptide derivative in an amount of 0.0001% to 10%, preferably in amount from 0.0001% to 1% based on the total weight of the composition.

The term “topical composition” as used herein refers in particular to cosmetic compositions that can be topically applied to mammalian keratinous tissue such as e.g. human skin or hair (including eyelashes, the eyebrows) or the nails, particularly human skin.

The term “cosmetic composition” as used in the present application refers to cosmetic compositions as defined under the heading “Kosmetika” in Römpp Lexikon Chemie, 10th edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4^{th} edition, 1992.

The term cosmetically acceptable carrier refers to all carriers and/or excipients and/or diluents conventionally used in topical compositions or compositions.

Preferably, the topical compositions are in the form of a suspension or dispersion in solvents or fatty substances, or alternatively in the form of an emulsion or micro emulsion (in particular of O/W- or W/O-type), PIT-emulsion, multiple emulsion (e.g. O/W/O- or W/O/W-type), pickering emulsion, hydrogel, alcoholic gel, lipogel, one- or multiphase solution or vesicular dispersion or other usual forms, which can also be applied by pens, as masks or as sprays. If the topical composition is or comprises an emulsion it can also contain one or more anionic, nonionic, cationic or amphoteric surfactant(s).

Preferred topical compositions are skin care compositions, and functional compositions.

Examples of skin care compositions are, in particular, body oils, body lotions, body gels, treatment creams, skin protection ointments, shaving compositions, such as shaving foams or gels, skin powders such as baby powder, moisturizing gels, moisturizing sprays, revitalizing body sprays, cellulite gels, face and/or body moisturizers, facial and/or body cleansers, face masks, anti acne compositions and/or peeling compositions.

Examples of functional compositions are cosmetic or pharmaceutical compositions containing active ingredients such as hormone compositions, vitamin compositions, vegetable extract compositions, anti-ageing compositions, and/or antimicrobial (antibacterial or antifungal) compositions without being limited thereto.

Topical compositions in accordance with the invention can be in the form of a liquid, lotion, a thickened lotion, a gel, a cream, a milk, an ointment, a paste, a powder, a make-up, or a solid tube stick and can be optionally be packaged as an aerosol and can be provided in the form of a mousse such as a aerosol mousse, a foam or a spray foam, a spray, a stick, a plaster, a cleanser, a soap, a wipe or a lyophilizate (such as the Pentapharm Dual Vial system).

The compositions used according to the invention are preferably formulated an oil-in-water or water-in-oil emulsion, water-in-silicone or silicone-in-water emulsion or as an aqueous serum or aqueous gel.

The cosmetic compositions used according to the invention have a pH in the range of 3-10, preferably in the range of pH of 4-8, most preferred in the range of pH 4-6.

In accordance with the present invention, the topical composition contains at least one tripeptide derivative as defined above, optionally in combination with further ingredients such as ingredients for skin lightening; tanning prevention; treatment of hyperpigmentation; preventing or reducing acne, wrinkles, lines, atrophy and/or inflammation; as well as topical anesthetics; antimicrobial and/or antifungal agents; chelators and/or sequestrants; anti-cellulites and slimming (e.g. phytanic acid), firming, moisturizing and energizing, self tanning, soothing, as well as agents to improve elasticity and skin barrier and/or UV-filter substances. The topical cosmetic compositions can also contain usual cosmetic adjuvants and additives, such as preservatives/antioxidants, fatty substances/oils, water, organic solvents, silicones, thickeners, softeners, emulsifiers, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention, are e.g. described in the CTFA Cosmetic Ingredient Handbook, Second Edition (1992) without being limited thereto.

The usual cosmetic adjuvants and additives such as e.g. emulsifiers, thickeners, surface active ingredients and film formers can show synergistic effects which can be determined by the expert in the field with normal trials, or with the usual considerations regarding the formulation of cosmetic composition.

The necessary amounts can, based on the desired product, easily be determined by the skilled person. The cosmetically active ingredients useful herein can in some instances provide more than one benefit or operate via more than one mode of action.

If nothing else is stated, the carrier, excipients, additives, diluents, adjuvant and additives etc. mentioned in the following are in particular suitable for topical compositions according to the present invention.

The topical compositions according to the present invention may contain further cosmetically active ingredients. Examples of cosmetically active ingredients comprise peptides and/or oligopeptides (such as e.g., Matrixyl™ [pentapeptide derivative], one or both of the peptides contained in SYN®-TACKS, SYN®-COLL (INCI: Palmitoyl Tripeptide-5, Glycerin), SYN®-AKE (INCI: Water, Glycerin, Dipeptide Diaminobutyric acid benzylamide diacetate (from DSM Nutritional Products Ltd., Branch Pentapharm), Ac-Gln-Asp-Val-His-OH and/or H-Lys-Asp-Val-Cit-NH2*2TFA), wax-based synthetic peptides and palmitoyl-oligopeptides, iodopropyl butylcarbamate, glycerol, urea, guanidine (e.g. amino guanidine); vitamins and derivatives thereof such as vitamin C (ascorbic acid), vitamin A (e.g., retinoid derivatives such as retinyl palmitate or retinyl propionate), vitamin E (e.g., tocopherol acetate), vitamin B₃ (e.g. niacinamide) and vitamin B₅ (e.g. panthenol), vitamin B₆ and vitamin B₁₂, biotin, folic acid; anti-acne actives or medicaments (e.g. resorcinol, salicylic acid, and the like); antioxidants (e.g. phytosterols, lipoic acid); flavonoids (e.g. isoflavones, phytoestrogens); skin soothing and healing agents such as aloe vera extract, allantoin and the like; agents suitable for aesthetic purposes such as essential oils, fragrances, skin sensates, opacifiers, aromatic compounds (e.g., clove oil, menthol, camphor, eucalyptus oil, and eugenol and their derivatives), desquamatory actives, hydroxy acids such as AHA acids, BHA acids, poly unsaturated fatty acids, radical scavengers, farnesol, antifungal actives in particular bisabolol, alkyldiols such as 1,2-pentanediol, hexanediol or 1,2-octanediol, phytol, polyols such as phytanetriol, ceramides and pseudoceramides, amino acids, protein hydrolysates, polyunsaturated fatty acids, plant extracts like kinetin, DNA or RNA and their fragmentation products, carbohydrates, conjugated fatty acids, carnitin, carnosine, biochinonen, phytofluen, phytoen, and their corresponding derivatives, co-enzyme Q10/ubiquinone), anti-oxidants such as preferably (−)-epigallocatechin gallate (EGCG), hydroxytyrosol and/or olive extract, shea butter, algae extract, cocoa butter, aloe extract, hyaluronic acid and elastin without being limited thereto.

Preferred examples of cosmetically active ingredients are vitamin C (ascorbic acid) and/or its derivatives (e.g. ascorbyl phosphate such as Stay C (sodium ascorbyl monophosphate) from DSM Nutritional Products Ltd.), vitamin A and/or its derivatives (e.g., retinoid derivatives such as retinyl palmitate or retinyl propionate), vitamin E and/or its derivatives (e.g., tocopherol acetate), vitamin B₆, vitamin B₁₂, biotin, co-enzyme Q10, EGCG, hydroxytyrosol and/or olive extract, shea butter, algae extract, cocoa butter, aloe extract, jojoba oil, echinacea extract, elastin and hyaluronic acid.

The additional cosmetically active ingredient is typically included in an amount of at least 0.001 wt. % based on the total weight of the topical composition. Generally, an amount of about 0.001 wt. % to about 30 wt. %, preferably from about 0.001 wt. % to about 10 wt. % of an additional cosmetically active agent is used.

Vitamin C (ascorbic acid) and/or its derivatives in particular ascorbyl phosphate such as Stay C (sodium ascorbyl monophosphate) is preferably used in the topical compositions according to the invention in an amount of 0.1-5 wt.-% in particular 0.1-2 wt.-%.

Shea butter is preferably used in the topical compositions according to the invention in an amount of 0.5-10 wt.-%, in particular 0.5-5 wt.-%.

Algae extract is preferably used in the topical compositions according to the invention in an amount of 0.1-10 wt.-%, in particular 0.5-1 wt.-%.

Aloe extract is preferably used in the topical compositions according to the invention in an amount of 0.1-10 wt.-%, in particular 0.5-1 wt.-%.

Elastin is preferably used in the topical compositions according to the invention in an amount of 0.01-10 wt.-%, preferably 0.01-1 wt.-%

A vitamin E derivative for use in the present invention is tocopheryl acetate. Tocopheryl acetate may be present in the topical compositions in an amount from about 0.05 wt.-% to about 25 wt.-%, in particular 0.05 wt.-% to 5 wt.-%. Another vitamin E derivative of interest is tocopheryl linoleate. Tocopheryl linoleate may be present in the skin care composition in an amount from about 0.05 wt.-% to about 25 wt.-% in particular 0.05 wt.-% to 5 wt.-%. Please verify

Vitamin A and/or its derivatives in particular retinoid derivatives such as retinyl palmitate or retinyl propionate is preferably used in the topical compositions according to the invention in an amount of 0.01-5 wt.-%, in particular 0.01-0.3 wt.-%

Cocoa butter is preferably used in the topical compositions according to the invention in an amount of 0.5-5 wt.-%.

Hyaluronic acid is preferably used in the topical compositions according to the invention combination in combination with a tripeptide as such a combination is able to effectively protect the skin in the inner and outer layers. Hyaluronic acid is preferably used in an amount of 0.1-0.5 wt.-%. Most preferably, the Hyaluronic acid is incorporated as a 1% solution of Hyaluronic Acid-BT which is e.g. commercially available from DSM Nutritional Products Ltd., Branch Pentapharm under the tradename Hyasol®-BT.

If not otherwise stated, the wt.-% indications are always based on the total weight of the composition.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

Which amount of the topical composition has to be applied, depends on the concentration of the active ingredient(s) in the product and the desired cosmetic effect(s). A typical “leave-on” composition like a skin care emulsion or a functional composition, for example, is usually applied in an amount of about 0.5 to about 2 mg per cm² skin. The applied amount is normally not critical, and the desired effect(s) may be achieved by using more of the composition, repeating the application of the composition and/or applying a composition which contains more of the active ingredient(s).

By “‘leave-on’ composition” as used herein a topical composition is meant which after having applied to the skin, is not removed intentionally. It is preferably left on the skin for a period of at least about 15 minutes, more preferably at least about 30 minutes, even more preferably at least about 1 hour, most preferably for at least several hours, e.g. up to about 12 hours.

In another embodiment, the invention also relates to a method of tightening, firming and/or moisturizing the skin said method comprising the step of applying an effective amount of a topical composition with all the definition and preferences as given above to the skin of a subject in need of such a treatment. In particular, the invention relates to a method of treatment or co-treatment of skin sagging, of maintaining or restoring the suppleness and elasticity of the skin, of facilitating cicatrization and of repairing epidermal micro-traumas or of reducing stretch marks said method comprising the step of applying an effective amount of a topical composition with all the definition and preferences as given above to the skin of a subject in need of such a treatment. The term treatment or co-treatment as used in the present invention includes also a proactive use of the topical compositions in order to prevent skin sagging, cicatrization or stretch marks.

An effective amount of a topical composition with the definitions and preferences as given above in these methods refers to an amount necessary to obtain a physiological effect. The physiological effect may be achieved by one single dose or by repeated doses. The dosage administered may, of course, vary depending upon known factors, such as the physiological characteristics of the particular composition and its mode and route of administration; the age, health and weight of the recipient; the nature and extent of the symptoms; the kind of concurrent treatment; the frequency of treatment; and the effect desired and can be adjusted by a person skilled in the art. Preferably, the topical compositions are applied at least twice a day such as e.g. once in the morning and once in the evening.

The following examples are provided to further illustrate the compositions and effects of the present invention. These examples are illustrative only and are not intended to limit the scope of the invention in any way.

### EXAMPLE 1

### Studies of the Effect of Tripeptide Derivatives on Synthesis of Hyaluronic Acid

### Measurement of Hyaluronan Synthesis in Normal Human Fibroblasts (NHF):

Normal human fibroblasts (NHF) were seeded in 96 well cell culture plates (Nunclon). After three days of growth a tripeptide derivative of the present invention was added in medium without FCS and the cells were incubated for an additional three days. Secreted Hyaluronan synthesis in the growth medium was measured using the Hyaluronan Assay Kit from Echelon (Salt Lake City, Utah).

### EXAMPLE 2

### Studies of the Effect of Tripeptide Derivatives on Synthesis of the Proteoglycan “Decorin”

### Measurement of Decorin Synthesis in Normal Human Fibroblasts:

Normal human fibroblasts (NHF) were grown in 96 well cell culture plates (Nunclon). After three days a tripeptide derivative of the present invention was added in medium without FCS and the cells were incubated for an additional three days. To measure Decorin synthesis an enzyme linked immunoassay (EIA) was performed. In brief: the cells were fixed with 4% paraformaldehyde, and permeabilized with 0.5 Triton-X100 followed by blocking of unspecific binding sites with 5% skim milk. To stain for Decorin the cells were incubated with goat-anti-Decorin antibody (R&Dsystems) which was detected with rabbit-anti-goat HRP conjugated (Pierce) secondary antibody. After addition of substrate Decorin synthesis was measured at 492 nm in an absorbance plate reader (Multiskan Ascent, Thermolabsystems).

### EXAMPLE 3

### Studies of the Effect of Tripeptide Derivatives on Synthesis of the Proteoglycan “Lumican”

### Measurement of Lumican Synthesis in Normal Human Fibroblasts:

Normal human fibroblasts (NHF) were grown in 96 well culture plates (Nunclon). After three days a tripeptide derivative of the present invention was added in medium without FCS and the cells were incubated for an additional three days. To measure Lumican synthesis an enzyme linked immunoassay (EIA) was performed. In brief: the cells were fixed with 4% paraformaldehyde, and permeabilized with 0.5 Triton-X100 followed by blocking of unspecific binding sites with 5% skim milk. To stain for Lumican the cells were incubated with goat-anti-Lumican (L-20) antibody (Santa Cruz Biotechnology, sc-27718) which was detected with goat-anti-mouse HRP conjugated (Pierce) secondary antibody. After addition of substrate Lumican synthesis was measured at 492 nm in an absorbance plate reader (Multiskan Ascent, Thermolabsystems).

The results were evaluated relative to a control consisting of cells that have not been treated with the tripeptide derivative of formula (I) and which is set to 100%.

TFA means the tripeptide derivative exists in the form of a trifluoroacetate salt.

n.t.=not tested

The results are reported in the following table:

TABLE 1

| | | Conc. | Synthesis Stimulation of | | |
|---|---|---|---|---|---|
| No | Tripeptide derivative* | [μM] | Hyaluronan | Decorin | Lumican |
| 1 | Palm-Lys-Val-Lys-OH *2 TFA | 10 | 220% | 200%/25 μM | 120% |
| 2 | Palm-Orn-Val-Lys-OH *2 TFA | 50 | 180% | n.t. | n.t. |
| 3 | Palm-Lys-Leu-Lys-OH *2 TFA | 25 | 180% | n.t. | n.t. |
| 4 | Palm-Lys-Ile-Dab-OH *2TFA | 25 | 180% | n.t. | n.t. |
| 5 | Palm-Lys-Nva-Dab-OH *2TFA | 25 | 220% | n.t. | n.t. |
| 6 | Palm-Lys-Leu-Dab-OH *2TFA | 50 | 220% | n.t. | n.t. |
| 7 | Palm-Lys-Val-Dab-NH-Hexadecyl | 25 | 300% | n.t. | n.t. |
| | *2 TFA | | | | |
| 8 | Tetradecyl-NH—C(O)-Dab-Val-Dab- | 12.5 | 400% | 170% | 165%/100 μM |
| | OH *2 TFA | | | | |
| 9 | Tetradecyl-NH—C(O)-Lys-Ile-Dab- | 25 | 220% | n.t. | n.t. |
| | OH *2 TFA | | | | |
| 10 | Tetradecyl-NH—C(O)-Arg-Val-Arg- | 25 | 220% | n.t. | n.t. |
| | OH *2 TFA | | | | |
| 11 | Myristoyl-Lys-Val-Lys-OH*2TFA | 100 | inactive | inactive | inactive |
| 12 | Lauroyl-Lys-Val-Lys-OH*2TFA | 100 | inactive | inactive | inactive |
| 13 | Caprinoyl-Lys-Val-Lys-OH*2TFA | 100 | inactive | inactive | inactive |
| 14 | Stearoyl-Lys-Val-Lys-OH*2TFA | 100 | inactive | inactive | inactive |
| 15 | H-Lys-Val-Lys-OH *3 TFA | 100 | inactive | inactive | inactive |
| 16 | Palm-Lys-Val-Orn-OH *2 TFA | 100 | inactive | inactive | inactive |
| 17 | Palm-Lys-Val-Dap-OH *2 TFA | 100 | inactive | inactive | inactive |
| 18 | Palm-Lys-Val-Dab-OH *2 TFA | 100 | inactive | inactive | inactive |

*All amino acids exhibit the L-configuration

In the following, the term Tripeptide No 8 refers to Tetradecyl-NH—C(O)-Dab-Val-Dab-OH*2 TFA (table 1, entry 8, INCI: Tetradecyl Aminobutyroylvalylaminobutyric Urea Trifluoroacetate).

### EXAMPLE 4

### In Vivo Study

The remodeling effect (effect on sagging and double chin) and firming effect (i.e. effect on skin tonicity and skin firmness) of a composition comprising the tripeptide No 8 was assessed by a double blind parallel group study with 41 volunteers, twice daily applied. Measurements were taken after 56 days (D56) and 84 days (D84).

TABLE 2
composition for the in vivo study

| | | | placebo | sample |
|---|---|---|---|---|
| Phase | Ingredients | INCI Name | wt.-% | |
| A | | Isohexadecane | 6 | 6 |
| | Jojoba Oil | Simmondsia Chinensis | 4 | 4 |
| | | (Jojoba) Seed Oil | | |
| | Tegosoft CT | Caprylic/Capric | 4 | 4 |
| | | Triglyceride | | |
| B | Aristoflex | Ammonium | 1 | 1 |
| | AVC | Acryloyldimethyltaurate/VP | | |
| | | Copolymer | | |
| C | Glycerin | Glycerin | 3 | 3 |
| | Phenonip | Phenoxyethanol and | 0.8 | 0.8 |
| | | Methylparaben and Ethylparaben | | |
| | | and Butylparaben and | | |
| | | Propylparaben and | | |
| | | Isobutylparaben | | |
| | Water | Aqua | Ad 100 | |
| | Tripeptide | | — | 0.0025 |
| | No 8 | | | |
| pH | | | 4.85 | 4.8 |

Manufacturing Instruction
Add phase B to phase A, let it disperse for a short time.
Add phase D to phase C, and stir until it's dissolved
Add phase CD to phase AB under stirring and then homogenize (1 min).
pH measure and adjustment.

The remodeling effect of the face contour was assessed using 3D Primos Body®. The skin biomechanical properties (i.e. firming effect) were measured by using a Cutometer®.

### Result

### A) Remodeling Effect:

a noticeable remodeling effect characterized by a significant decrease in the double chin after 84 days of use (Verum −0.500 mm, p=0.047; Placebo: −0.214 mm) and by significant decreases in the ptose (sagging) in the face volume after 56 days of use (Verum −1.200 ml, p=0.04; Placebo: +1.342 ml).

### B) Firming Effect:

a firming effect characterized by the significant increase of the skin tonicity (17% on D56; improvement in 84% of the subjects) and of the skin firmness (+7% on D56; improvement in 84% of the subjects.

### EXAMPLE 5

### Water in Oil Cream

| Phase | Ingredients | INCI Name | % Wt. |
|---|---|---|---|
| A | Cremophor WO-7 | PEG-7 Hydrogenated Castor Oil | 2.50 |
| | Elfacos ST-9 | PEG-45/Dodecyl Glycol Copolymer | 2.00 |
| | Cirebelle 303 | Synthetic Wax | 5.00 |
| | Cirebelle 109L | Synthetic Wax | 7.20 |
| | Miglyol 818 | Caprylic/Capric/Linoleic Triglyceride | 5.00 |
| | Eutanol G | Octyldodecanol | 7.50 |
| | Cetiol OE | Dicaprylyl Ether | 8.20 |
| B | Deionised Water | Aqua | 52.30 |
| | Glycerine | Glycerin | 5.00 |
| | Propylene Glycol | Propylene Glycol | 2.00 |
| | Euxyl PE 9010 | Phenoxyethanol and | 0.80 |
| | | Ethylhexylglycerin | |
| | Magnesium | Magnesium Sulfate | 1.00 |
| | Sulfate | | |
| | Heptahydrate | | |
| C | Tripeptide No. 8 | | 0.0025 |

### EXAMPLE 6

### Formulation Containing Further Peptidic Ingredients

| Phase | Ingredients | INCI Name | % Wt. |
|---|---|---|---|
| A | Pemulen TR-1 | Acrylates/C10-30 Alkyl | 0.30 |
| | | Acrylate Crosspolymer | |
| | Cetiol 868 | Ethylhexyl Stearate | 8.00 |
| | Macadamianussöl | Macadamia Ternifolia Seed Oil | 8.00 |
| | Rapithix A 60 | Sodium Polyacrylate and | 1.00 |
| | | Hydrogenated Polydecene | |
| | | and Trideceth-6 | |
| | Euxyl 9010 | Phenoxyethanol and | 0.80 |
| | | Ethylhexylglycerin | |
| | Glycerine 86% | Glycerin | 2.00 |
| | Deionised Water | Aqua | ad 100 |
| B | SYN ®-COLL | Palmitoyl Tripeptide-5, Glycerine | 1.50 |
| | SYN ®-TACKS | Glycerin (and) Palmitoyl Dipeptide-5 | 0.80 |
| | | Diaminobutyloyl Hydroxythreonine | |
| | | (and) Palmitoyl Dipeptide-6 | |
| | | Diaminohydroxybutyrate | |
| | SYN ®-AKE | Water (and) Glycerin (and) Dipeptide | 3.00 |
| | | Diaminobutyroyl Benzylamide | |
| | | Diacetate | |
| | Tripeptide No. 8 | | 0.0015 |
| C | Parfum | Fragrance | 0.10 |

### EXAMPLE 7

### Anti Dark Circle Eve Cream

| Phase | Ingredients | INCI Name | % Wt. |
|---|---|---|---|
| A | Emulgade PL 68/50 | Cetearyl Glucoside and | 2.00 |
| | | Cetearyl Alcohol | |
| | Lanette E | Sodium Cetearyl Sulfate | 0.25 |
| | Tegin 4100 Pellets | Glyceryl Stearate | 1.00 |
| | Cetiol OE | Dicaprylyl Ether | 4.00 |
| | Shea Butter | Butyrospermum Parkii | 2.00 |
| | Cetiol PGL | Hexyldecanol (and) Hexyldecyl | 3.00 |
| | | Laurate | |
| | Jojobaöl | Jojobaoil | 1.00 |
| | DC 345 | Cyclopentasiloxane and | 0.50 |
| | | Cyclohexasiloxane | |
| | Rapithix A 100 | Sodium Polyacrylate | 0.50 |
| | Parsol SLX | Polysilicone-15 | 1.00 |
| B | Deionised Water | Aqua | ad 100 |
| | Glycerine | Glycerin | 2.00 |
| | Euxyl PE 9010 | Phenoxyethanol and | 1.00 |
| | | Ethylhexylglycerin | |
| | Xirona Magic Mauve | Silica C.I. 77891 Tin Oxide | 5.00 |
| C | Tripeptide No 8 | | 0.0015 |
| D | NaOH 10% | Sodium Hydroxide | q.s. |

### EXAMPLE 8

### Face Sculptor Restructuring Lift Cream

| Phase | Ingredients | INCI Name | % Wt. |
|---|---|---|---|
| A | Emulgade PL 68/50 | Cetearyl Glucoside and Cetearyl | 7.00 |
| | | Alcohol | |
| | Cutina GMS | Glyceryl Stearate | 4.00 |
| | Lanette E | Sodium Cetearyl Sulfate | 1.00 |
| | Shea Butter | Butyrospermum Parkii | 13.00 |
| | Cetiol CC | Dicaprylyl Carbonate | 3.00 |
| | Cetiol OE | Dicaprylyl Ether | 3.00 |
| | Tegosoft MM | Myristyl Myristate | 0.50 |
| | DC 345 | Dimethicone | 1.00 |
| | Eutanol G | Octyldodecanol | 1.00 |
| | Rapithix A 60 | Sodium Polyacrylate | 0.50 |
| | Euxyl PE 9010 | Phenoxyethanol and | 0.80 |
| | | Ethylhexylglycerin | |
| B | Deionised Water | Aqua | ad 100 |
| | Glycerine | Glycerin | 6.00 |
| C | Tripeptide No 8 | | 0.0015 |
| D | NaOH 10% | Sodium Hydroxide | q.s. |

### EXAMPLE 9

### Facial Cream

| Phase | Ingredients | INCI Name | % Wt. |
|---|---|---|---|
| A | Imwitor 372P | Glyceryl Stearate Citrate | 2.00 |
| | Cutina GMS | Glyceryl Stearate | 3.00 |
| | Sympatens-O/4200 | Sorbitan Laurate, Polyglyceryl-10 | 1.00 |
| | Sweet Almond Oil | Prunus Amygdalus Dulcis | 2.50 |
| | | (Sweet Almond) Oil | |
| | Tegosoft TN | C12-15 Alkyl Benzoate | 7.00 |
| | Cetiol OE | Dicaprylyl Ether | 5.00 |
| | Tegosoft DC | Decyl Cocoate | 3.00 |
| | BHT | BHT | 0.05 |
| | Euxyl PE 9010 | Phenoxyethanol, | 1.00 |
| | | Ethylhexylglycerin | |
| | Dow Corning 345 | Cyclomethicone | 2.00 |
| B | Keltrol RD | Xanthan Gum | 0.30 |
| | Glycerine | Glycerin | 2.00 |
| | Deionised Water | Aqua | ad 100 |
| | Tripeptide No 8 | | 0.0025 |
| | Rapithix A-60 | Sodium Polyacrylate and | 0.30 |
| | | Hydrogenated | |
| | | Polydecene and Trideceth-6 | |
| C | Acid or Base if | | |
| | necessary to pH 5-6 | | |

### EXAMPLE 10

### Concealer

### Silicone Based

| Phase | Ingredients | INCI Name | % Wt. |
|---|---|---|---|
| A | DC 9701 | Dimethicone/Vinyl Dimethicone | 3.00 |
| | | Crosspolymer and Silica | |
| | Cetiol CC | Dicaprylyl Carbonate | 2.00 |
| | DC 556 Fluid | Phenyl Trimethicone | 2.40 |
| | Lexfeel 7 | Neopentyl Glycol Diheptanoate | 3.40 |
| B | DC 9040 | Cyclopentasiloxane, Dimethicone | ad 100 |
| | | Crosspolymer | |
| | DC BY 11-030 | Cyclopentasiloxane | 5.00 |
| | | and PEG/PPG-19/19 Dimethicone | |
| | DC 345 | Cyclopentasiloxane and | 3.00 |
| | | Cyclohexasiloxane | |
| | Parsol SLX | Polysilicone-15 | 5.00 |
| | Euxyl 9010 | Phenoxyethanol and | 0.80 |
| | | Ethylhexylglycerin | |
| C | Tripeptide No 8 | | 0.0025 |
| | Deionised Water | Aqua | 0.75 |
| | Glycerine | Glycerin | 1.75 |

### EXAMPLE 11

### Good Morning Cream

| Phase | Ingredients | INCI Name | % Wt. |
|---|---|---|---|
| A | Olivem 1000 | Cetearyl Olivate (and) Sorbitan | 4.00 |
| | | Olivate | |
| | Merquat Plus | Polyquaternium 39 | 2.00 |
| | 3330 | | |
| | Fitoderm | Squalane | 5.00 |
| | Abil-350 | Dimethicone | 0.50 |
| | Tegosoft CT | Caprylic/Capric Triglyceride | 4.00 |
| | Lexfeel 7 | Neopentyl Glycol Diheptanoate | 3.00 |
| B | Deionised Water | Aqua | ad 100 |
| | Preservative | | q.s. |
| | Glycerin | Glycerin | 5.00 |
| | Structure XL | Hydroxypropyl Starch Phosphate | 0.90 |
| C | Spherica P-1500 | Silica | 1.00 |
| | Tripeptide No 8 | | 0.002 |
| | PEPHA ®-CTIVE | Water, Algae Extract | 3.00 |

### EXAMPLE 12

### Oil in Water Foundation

| Phase | Ingredients | INCI Name | % Wt. |
|---|---|---|---|
| A | Deionised Water | Aqua | ad 100 |
| | Glycerine | Glycerin | 2.00 |
| | Triethanolamine 99% | Triethanolamine | 0.80 |
| | Paratexin M | Methylparaben EP | 0.20 |
| | Keltrol RD | Xanthan Gum | 0.30 |
| | Tripeptide No 8 | | 0.003 |
| B | SOFT-TEX Titanium | C.I. 77891 | 4.57 |
| | Dioxide White C47- | | |
| | 7756 | | |
| | SunCROMA Yellow | C.I. 77492 | 0.30 |
| | Iron Oxide C33-1700 | | |
| | SunCROMA Red | C.I. 77491 | 0.13 |
| | Iron Oxide C33-2199 | | |
| | SunCROMA Black | C.I. 77499 | 0.20 |
| | Iron Oxide C33-5000 | | |
| C | DC 556 | Phenyl Trimethicone | 3.60 |
| | Dervacid 3155 Flake | Stearic Acid | 1.40 |
| | Nacol 16-95 | Cetyl Alcohol | 3.00 |
| | Paratexin P | Propylparaben EP | 0.10 |

### EXAMPLE 12

### Bubble Hair and Shower

| Phase | Ingredients | INCI Name | % Wt. |
|---|---|---|---|
| A | Deionised Water | Aqua | Ad 100 |
| | Carbopol Aqua SF- | Acrylates Copolymer | 7.50 |
| | 1 Polymer | | |
| | Texapon NSO-BZ | Sodium Laureth Sulfate | 40.00 |
| B | Miranol Ultra C 32 | Sodium Cocoamphoacetate | 5.00 |
| | Hostapon CLG | Sodium Lauroyl Glutamate | 4.50 |
| C | Jaguar C 162 | Hydroxypropyl Guar | 10.00 |
| | (pre-mix 2%) | Hydroxypropyltrimonium Chloride | |
| | Tripeptide No. 8 | | 0.0025 |
| | Euxyl K 300 | Phenoxyethanol & | 0.80 |
| | | Methylparaben & Propylparaben & | |
| | | Ethylparaben & Butylparaben & | |
| | | Isobutylparaben | |
| | Parfum Limette | Fragrance | 0.50 |
| | FD&C Yellow 5 | CI 19140 | q.s. |
| | Frescolat Plus | Menthyl Lactate, Menthol | 0.20 |
| D | Dehyton AB-30 | Coco Betaine | 2.00 |
| | Rewoderm LI S 80 | PEG-200 Hydrogenated Glyceryl | 1.00 |
| | | Palmate & PEG-7 Glyceryl | |
| | | Cocoate | |
| | Citric Acid | Citric Acid | q.s. |

### EXAMPLE 13

### Lip Volume Gloss

| Phase | Ingredients | INCI Name | % Wt. |
|---|---|---|---|
| A | Versagel ME 750 | Hydrogenated Polyisobutene, | ad 100 |
| | | Ethylene/Propylene/ | |
| | | Styrene Copolymer, Butylene/ | |
| | | Ethylene/Styrene Copolymer | |
| | Colorona Bordeaux | Iron Oxides, Mica | 5.00 |
| B | SYN ®-COLL | Glycerin, Palmitoyl Tripeptide-5 | 2.50 |
| | Tripeptide No. 8 | | 0.004 |
| | Poly-Pore E200 | Allyl Methacrylate Crosspolymer | 0.35 |

### EXAMPLE 14

### Mascara

| Phase | Ingredients | INCI Name | % Wt. |
|---|---|---|---|
| A | Dervacid 3155 | Stearic Acid | 4.00 |
| | Flake | | |
| | Glyceryl Stearate | Glyceryl Stearate SE | 4.00 |
| | SE | | |
| | Cirebelle 303 | Synthetic Wax | 5.00 |
| | Cirebelle 109L | Synthetic Wax | 2.00 |
| | Miglyol 808 | Caprylic/Capric/Linoleic | 5.00 |
| | | Triglyceride | |
| B | Deionised Water | Aqua | 49.80 |
| | Covapate Uniblack | C.I. 77499, Ricinus Communis | 10.00 |
| | | (Castor) Seed Oil | |
| | Triethanolamine | Glyceryl Rosinate, Octyldodecyl | 1.20 |
| | 99% | | |
| | Paratexin FPX | Myristate Triethanolamine | 1.00 |
| | | Phenoxyethanol, Methylparaben, | |
| | | Ethylparaben, Butylparaben, | |
| | | Propylparaben, Isobutylparaben | |
| C | Syntran 5190 | Acrylates Copolymer, Propylene | 18.00 |
| | | Glycol, Sodium Laureth-12 | |
| | | A Sulfate, qua, Methylparaben, | |
| | | Propylparaben, EDTA, Potassium | |
| | | Sorbate | |
| | Tripeptide No. 8 | | 0.003 |

### EXAMPLE 15

### Alcohol Free Facial Tonic

| Phase | Ingredients | INCI Name | % Wt. |
|---|---|---|---|
| A | Protasorb L-20 | Polysorbate 20 | 2.00 |
| | Alpaflor Calendula | Calendula Officinalis Extract, | 0.80 |
| | | Glycerin, Water | |
| | Alpaflor Buddleja | Buddleja Davidii Extract, | 0.80 |
| | | Glycerin, Water | |
| | Arlasilk | Sodium Coco PG-Dimonium | 0.50 |
| | | Chloride Phosphate | |
| | Phospholipd CDM | | |
| | Fragrance | Parfum | 0.10 |
| B | Deionised Water | Aqua | 93.19 |
| | Citric Acid | Citric Acid | 0.01 |
| | Monohydrate | | |
| | Tripeptide No. 8 | | 0.0035 |
| | Paratexin FPX | Ethylparaben, Butylparaben, | 0.10 |
| | | Propylparaben, Isobutylparaben | |

### EXAMPLE 15

### Leave-in Hair and Scalp Conditioner

| Phase | Ingredients | INCI Name | % Wt. |
|---|---|---|---|
| A | Deionised Water | Aqua | ad 100 |
| | Ethanol DEB 96 | Alcohol denat. | 30.00 |
| | PVP/VA Copolymer | PVP/VA Copolymer | 2.50 |
| | Euxyl K-300 | Phenoxyethanol, | 0.80 |
| | | Methylparabene, Butyl- | |
| | | parabene, Ethylparabene, | |
| | | Propylparabene, | |
| | | Isobutylparabene | |
| B | Protachem HCO-40 | PEG-40 Hydrogenated Castor | 0.50 |
| | | Oil | |
| | Fragrance | Parfum | 0.10 |
| C | Triethanolamine | Triethanolamine | 0.01 |
| | 99% | | |
| D | FD & C Yellow No 5 | CI 19140, Aqua | 0.10 |
| | (0.5% Solution) | | |
| | FD & C Blue No 1 | CI 42090, Aqua | 0.10 |
| | (0.5% Solution) | | |
| | Tripeptide No 8 | | 0.0045 |
| | Deionised Water | Aqua | 0.75 |
| | Glycerine | Glycerin | 1.75 |

1. Use of a tripeptide derivative of the general formula (I)
R1-A-B-C-R2 (I),
wherein
R1 means palmitoyl or tetradecylaminocarbonyl;
R2 means OH or NH-hexadecyl;
A means argininyl, lysyl, ornithyl or 2,4-diaminobutyroyl;
B means valyl, leucyl, isoleucyl or norvalyl and
C means argininyl, lysyl or 2,4-diaminobutyroyl
wherein the tripeptide derivative is selected from the group of Palm-Lys-Val-Lys-OH, Palm-Orn-Val-Lys-OH, Palm-Lys-Leu-Lys-OH, Palm-Lys-Ile-Dab-OH, Palm-Lys-Nva-Dab-OH, Palm-Lys-Leu-Dab-OH, Palm-Lys-Val-Dab-NH-Hexadecyl, Tetradecyl-NH—C(O)-Dab-Val-Dab-OH, Tetradecyl-NH—C(O)-Lys-Ile-Dab-OH, Tetradecyl-NH—C(O)-Arg-Val-Arg-OH or the dermatologically tolerated salts thereof for tightening, firming and/or moisturizing the skin.

2. Use according to claim 1 for lifting the skin, preventing or decreasing skin sagging, maintaining or restoring the suppleness and elasticity of the skin, facilitating cicatrization, repairing epidermal micro-traumas and/or reducing stretch marks.

3. Use according to claim 1, wherein A and C, which may be identical or different, have the definitions of lysyl or 2,4-diaminobutyroyl.

4. Use according to claim 1, wherein R2 represents OH.

5. Use according to claim 1, wherein the tripeptide derivative is Tetradecyl-NH—C(O)-Dab-Val-Dab-OH in the form of the trifluoroacetate salt.

6. Use according to claim 1, wherein all amino acids of the tripeptide derivative are L-configurated.

7. Use according to claim 1, wherein an effective amount of at least one tripeptide derivative is comprised in a topical composition further comprising a cosmetically acceptable carrier.

8. Use according to claim 7, wherein the topical composition further comprises 0.1 to 0.5 wt.-% of hyaluronic acid.

9. A method of tightening, firming and/or moisturizing the skin said method comprising the step of applying an effective amount of a topical composition comprising an effective amount of a tripeptide derivative as defined in claim 1 and a cosmetically acceptable carrier to the skin of a subject in need of such a treatment.

10. A method according to claim 9 for the treatment or co-treatment of skin sagging, for maintaining or restoring the suppleness and elasticity of the skin, for facilitating cicatrization, for repairing epidermal micro-traumas and/or for reducing stretch marks.

11. A method for stimulating the synthesis of glycosaminoglycans containing a D-glucosamine and/or N-acetyl-D-glucosamine residue and/or proteoglycans by fibroblasts and/or keratinocytes, comprising administering to an individual in need of such treatment, an effective amount of at least one tripeptide derivative corresponding to formula (I) below:
R1-A-B-C-R2 (I),
in which R1 is palmitoyl or tetradecylaminocarbonyl; R2 is OH or NH-hexadecyl; A is argininyl, lysyl, ornithyl or 2,4-diaminobutyroyl; B is valyl, leucyl, isoleucyl or norvalyl and C is argininyl, lysyl or 2,4-diaminobutyroyl or the dermatologically tolerated salts thereof.

12. The method according to claim 11 comprising stimulating the synthesis of Hyaluronic acid and/or of the proteoglycans Decorin and/or Lumican.
